(19) United States
(12) Patent Application Publication
(10) Pub. No.: US 2015/0259348 A1
(43) Pub. Date: Sep. 17, 2015

(54) PROCESS FOR PREPARING PEMETREXED DI POTASSIUM AND ITS HYDRATES

(71) Applicant: SHILPA MEDICARE LIMITED, RAICHUR KARNATAKA (IN)

(72) Inventors: Vimal Kumar Sharawat, Raichur (IN); Dr. Rafiuddin, Raichur (IN); Vinod Kumar Singh, Raichur (IN); Bhagat Raj Pipal, Raichur (IN); Akshay Kant Chaturvedi, Raichur (IN)

(73) Assignee: SHILPA MEDICARE LIMITED, RAICHUR (IN)

(21) Appl. No.: 14/426,765

(22) PCT Filed: Oct. 16, 2013
(86) PCT No.: PCT/IB2013/059378

§ 371 Date: Mar. 9, 2015

(30) Foreign Application Priority Data
Oct. 17, 2012 (IN) 4322/CHE/2012

(51) Int. Cl. C07D 487/04

(52) U.S. Cl. CPC C07D 487/04

(57) ABSTRACT

The present invention provides Di potassium (S)-2-(4-(2-(2-amino-4-oxo-4,7-dihydro-3H-pyrrolo[2,3-d]pyrimidin-5-yl)ethyl)benzamido)pentanedioate (I)

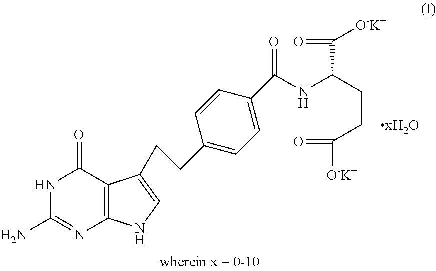

and its hydrates useful as active pharmaceutical ingredient in pharmaceutical compositions for the treatment of cancer. The present invention also provides process for preparation thereof.

The present application is a US National stage patent application of PCT/IB2013/059378, filed Oct. 16, 2013, which claims priority to IN Patent Application No. 4322/CHE/2012, filed Oct. 17, 2012, the disclosures of each of which are incorporated by reference herein in their entirety.

### INTRODUCTION

Pemetrexed's chemical name is (S)-2-(4-(2-(2-amino-4-oxo-4,7-dihydro-3H-pyrrolo[2,3-d]pyrimidin-5-yl)ethyl)benzamido)pentanedioic acid and has the following chemical structure:

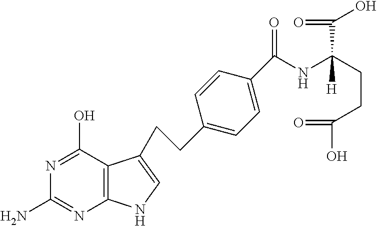

The most common salt of pemetrexed is a disodium salt, which is often referred as pemetrexed disodium. Pemetrexed disodium has the chemical name L-Glutamic acid, N-[4-[2-(2-amino-4,7-dihydro-4-oxo-1H-pyrrolo[2,3-d]pyrimidin-5-yl)-ethyl]benzoyl]-, disodium salt. Pemetrexed disodium heptahydrate is the active ingredient of Eli Lilly and Company's ALIMTA® injectable composition. Pemetrexed disodium heptahydrate has the following chemical structure:

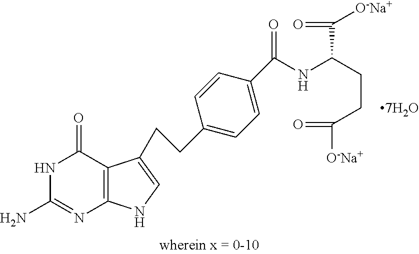

Pemetrexed disodium is a multi-targeted antifolate that strongly inhibits various folate-dependent enzymes, including thymidylate synthase (TS), dihydrofolate reductase (DHFR) and glycinamide ribonucleotide formyltransferase (GARFT). Pemetrexed disodium has been proved effective on a wide variety of solid tumors in clinical trials. Currently, pemetrexed disodium is commercial available in USA, European Union, Canada, Japan and China etc. for treatment of malignant pleural stromal tumor as a first-line drug, and local advanced and metastatic non-small cell lung cancer as a second-line drug. In the treatment of malignant pleural stromal tumor, pemetrexed disodium is a unique chemotherapeutic agent in the market currently. In the second-line treatment of non-small cell lung cancer, pemetrexed disodium has a comparative efficacy and reduced toxicities compared with the standard drug Docetaxel. Hence, it is likely for pemetrexed disodium to become a new standard treatment of the second-line treatment for non-small cell lung cancer. In addition, the clinical studies of pemetrexed disodium in the treatment of breast, bowel, pancreatic, head and neck, gastric and bladder cancers are still ongoing.

Various methods for preparing pemetrexed and pemetrexed disodium are disclosed in the prior art, such as W02001014379A, WO1999016742, EP432677, EP589720, WO0011004, EP549886 and CN1778797. Luo Jie et al in US20100305319A1 (equivalent: EP 2213674B1) describes a method of purifying a salt of pemetrexed have a structure of formula (III)

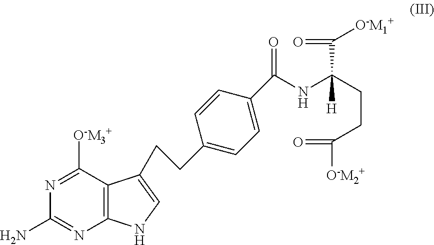

by salting-out, wherein if M₃⁺ is H⁺, then each of M₁⁺ and M₂⁺ is independently H⁺, Li⁺, Na⁺ or K⁺, provided that both of them are not H⁺; if M₃⁺ is Li⁺, Na⁺ or K⁺, then each of M₁⁺ and M₂⁺ is independently Li⁺, Na⁺ or K⁺. In example 16 and 17, it provides mention of purification of potassium pemetrexed, however, it appears that it does not refer to other than mono potassium salt of pemetrexed with no characterization details of the said salt. Further, in our attempt to reproduce the said example disclosure, no material could be recovered.

Pemetrexed being an important anticancer therapeutic agent, additional and improved ways of preparing pemetrexed pharmaceutically acceptable salt may be of immense value to pharmaceutical science and the healthcare of cancer patients. Hence, there exists a need for the development of new salt in stable form and economically viable process, which may be industrially amenable to scalable up, economically viable, safer for handling, less time consuming and with better and consistent quality parameters.

### SUMMARY OF INVENTION

Particular aspects of the present application relate to the process for preparation of Pemetrexed di potassium (I) and its hydrates—

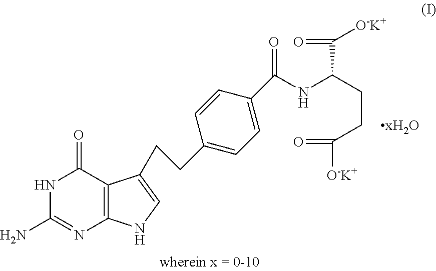

This application also relates to the process for preparation of Pemetrexed di potassium (I) and its hydrates, which is substantially free from process related impurities. Pemetrexed di potassium (I) and its hydrates obtained by the process according to the present invention are useful as active pharmaceutical ingredient in pharmaceutical compositions for the treatment of cancer. Different aspects of the present application are summarized herein below individually.

In one aspect of the present application, the present invention provides Di potassium (S)-2-(4-(2-(2-amino-4-oxo-4,7-dihydro-3H-pyrrolo[2,3-d]pyrimidin-5-yl)ethyl)benzamido)pentanedioate (I) and its hydrates.

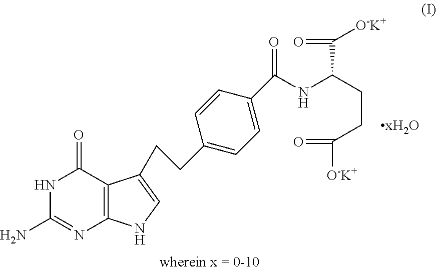

In another aspect of the present invention, it provides process for preparing Di potassium (S)-2-(4-(2-(2-amino-4-oxo-4,7-dihydro-3H-pyrrolo[2,3-d]pyrimidin-5-yl)ethyl)benzamido)pentanedioate (I) comprising reacting aqueous potassium hydroxide with starting material of Formula (A)

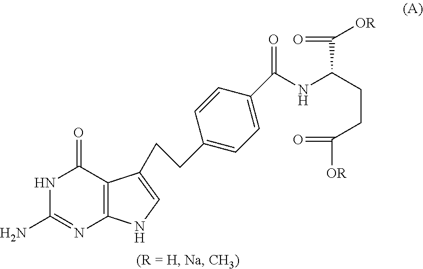

In a particular aspect of the present invention compound of Formula (A) i.e. starting material for the preparation of compound of Formula (I) is selected from N-[4-[2-(2-amino-4,7-dihydro-4-oxo-1H-pyrrolo[2,3-d]pyrimidin-5-yl)ethyl]benzoyl]-L-glutamic disodium salt or N-[4-[2-(2-amino-4,7-dihydro-4-oxo-1H-pyrrolo[2,3-d]pyrimidin-5-yl)ethyl]benzoyl]-L-glutamic di-acid or N-[4-[2-(2-amino-4,7-dihydro-4-oxo-1H-pyrrolo[2,3-d]pyrimidin-5-yl)ethyl]benzoyl]-L-glutamic dimethyl ester.

The compound of formula (I) according to the present invention is isolated as dipotassium salt of pemetrexed having purity greater than 99% w/w and total impurities are not more than 0.7% w/w. Said compound of formula (I) and its hydrates are useful as active pharmaceutical ingredient in pharmaceutical compositions for the treatment of cancer.

### DETAILED DESCRIPTION

As set forth herein, embodiments of the present invention provide Pemetrexed di potassium (I) and its hydrates and process for preparation thereof. Individual embodiments of the present invention are detailed herein below separately.

In one embodiment of the present application, it provides Pemetrexed di potassium (I) and its hydrates.

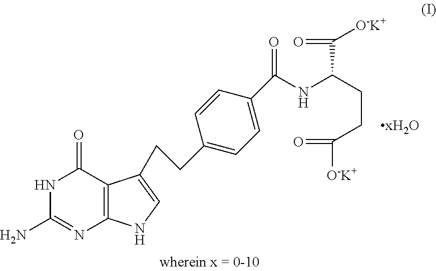

This application relates to process for preparation of Pemetrexed di potassium (I) and its hydrates, which is substantially free from process related impurities. Pemetrexed di potassium (I) and its hydrates obtained by the process according to the present invention are useful as active pharmaceutical ingredient in pharmaceutical compositions for the treatment of cancer. Different embodiments of the present application are detailed herein below individually. In one embodiment according to the present application, it provides Di potassium (S)-2-(4-(2-(2-amino-4-oxo-4,7-dihydro-3H-pyrrolo[2,3-d]pyrimidin-5-yl)ethyl)benzamido)pentanedioate (I) salt—

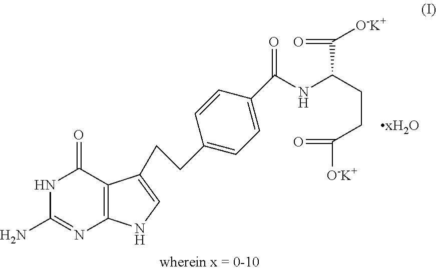

and its hydrates.

Dipotassium (S)-2-(4-(2-(2-amino-4-oxo-4,7-dihydro-3H-pyrrolo[2,3-d]pyrimidin-5-yl)ethyl)benzamido)pentanedioate (I) according to present invention is highly pure compound, wherein potassium content ranges between 14.5% to 16.5% w/w on anhydrous basis. Said Di potassium salt of pemetrexed having purity greater than 99% w/w and total impurities are not more than 0.7% w/w.

In another embodiment of the present invention, it provides process for preparing Di potassium (S)-2-(4-(2-(2-amino-4-oxo-4,7-dihydro-3H-pyrrolo[2,3-d]pyrimidin-5-yl)ethyl)benzamido)pentanedioate (I) comprising reacting with aqueous potassium hydroxide with starting material of Formula (A)

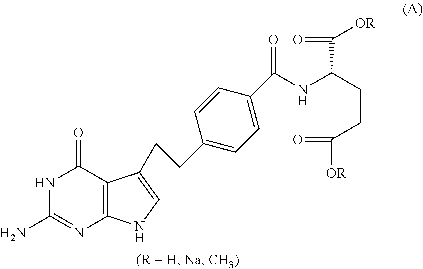

Compound of Formula (A) i.e. starting material for the preparation of compound of Formula (I) is selected from N-[4-[2-(2-amino-4,7-dihydro-4-oxo-1H-pyrrolo[2,3-d]pyrimidin-5-yl)ethyl]benzoyl]-L-glutamic disodium salt or N-[4-[2-(2-amino-4,7-dihydro-4-oxo-1H-pyrrolo[2,3-d]pyrimidin-5-yl)ethyl]benzoyl]-L-glutamic di-acid or N-[4-[2-(2-amino-4,7-dihydro-4-oxo-1H-pyrrolo[2,3-d]pyrimidin-5-yl)ethyl]benzoyl]-L-glutamic dimethyl ester. The process necessarily involves the use of alcohol solvent, which is selected from C1 to C5 alcohols including both branched or straight chain alkane.

Scheme-I provides summary of the process encompassed according to the present invention. The process is based on the choice of individual key starting materials; however, it can also be understood based on the non-limiting examples given later.

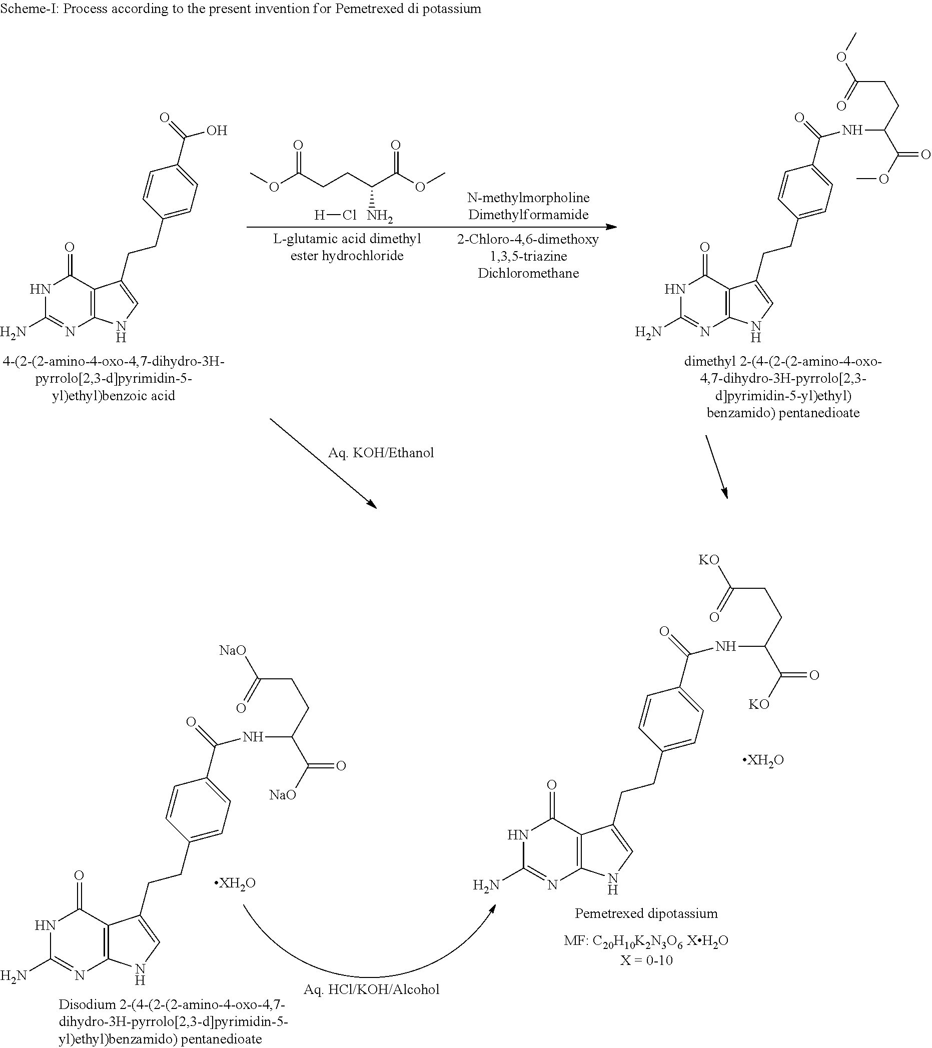

Overall process for preparing Di potassium (S)-2-(4-(2-(2-amino-4-oxo-4,7-dihydro-3H-pyrrolo[2,3-d]pyrimidin-5-yl)ethyl)benzamido)pentanedioate (I) comprises the steps of—
a) providing a solution of compound of Formula (A);

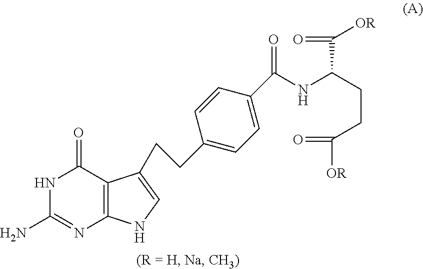

b) cooling the reaction mass between 0-15° C.;
c) adding aqueous solution of KOH under stirring at 0-15° C.;
d) stirring the reaction mass for 30-120 mins at 0-25° C.;
e) adjusting pH of aqueous reaction mass to 7.0-8.5 by using aqueous hydrochloric acid solution;
f) optionally performing the carbon treatment;
g) optionally adding a water soluble potassium salt to the reaction mixture;
h) cooling the reaction mass to 0-10° C. under stirring;
i) adding alcohol solvent at 0-10° C.;
j) optionally repeating the steps a) to g) at room temperature, by isolating the solid material from step i); and
k) isolating the solid product of formula (I).

In the step c) wherein aqueous solution of KOH is added under stirring at 0-15° C., it is necessary to ensure the temperature range to be at lower level, since addition of aqueous KOH done at temperature above 15° C. may result in large number of degradation impurities formation leading to recovery of an impure material, which is difficult to purify in the subsequent steps. The process for preparing compound of formula (I) involves use of alcohol in step i), wherein alcohol is selected from C1 to C5 alcohol. In one of the particular embodiment, it involved the use of ethanol. As per another embodiment of this application, Water soluble potassium salt used in step g) is preferably potassium acetate.

In process for preparing compound of formula (I) as discussed above, isolation of the solid title product comprise steps of—
a) filtering the solid;
b) washing with alcohol (C1-05) solvent; and
c) drying the solid to get compound of formula-(I) having purity greater than 99% w/w.

The drying is performed between temperatures ranging between 35-50° C. under high vacuum conditions (550 to 700 mmHg) to recover the desired stable hydrated material.

In another embodiment of the present invention, when group ‘R.’ of Formula (A) is Na or H, compound of Formula (A) is provided as solution in water solvent optionally containing hydrochloric acid; and when ‘R’ is CH₃ compound of Formula (A) is provided as solution in a water immiscible organic solvent selected from dichloromethane, toluene, hexane and heptane.

In a process for preparing Di potassium (S)-2-(4-(2-(2-amino-4-oxo-4,7-dihydro-3H-pyrrolo[2,3-d]pyrimidin-5-yl)ethyl)benzamido)pentanedioate (I) starting from pemetrexed disodium salt, the starting material i.e. pemetrexed disodium, is reacted with aqueous hydrochloric acid solution at temperature between 10-30° C. Retaining the pH between 2.0 to 4.0, reaction mixture is subjected to stirring, for time ranging between 1-4 hrs. On completion of the reaction, the reaction mass is filtered and optionally washed with water, which is followed by drying. The residue obtained is then combined with water and further proceeded to obtain compound of formula (I) by the process of the present invention.

In yet another embodiment, the process for preparing compound of formula (I) starting from pemetrexed dimethyl ester optionally involves the recovery of water immiscible organic solvent layer, re-addition of water immiscible organic solvent, stirring and separation of aqueous layer. Pemetrexed dimethyl ester, used in this reaction may be used in an already isolated form or in-situ form if prepared fresh from its starting materials.

In the process for preparing pemetrexed di potassium according to the present invention, KOH is utilized up to more than 2.2 Moles but less than 4 moles with respect to starting material i.e. compound of Formula (A). In some of the embodiment's amount of KOH used may vary from range of 2.2-4 moles depending upon the method of preparation of the starting materials of Formula (A).

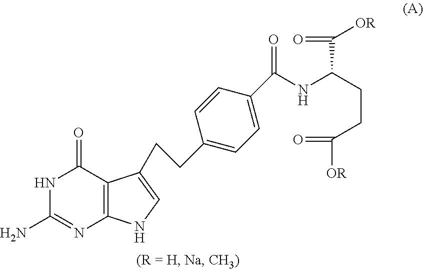

Di potassium (S)-2-(4-(2-(2-amino-4-oxo-4,7-dihydro-3H-pyrrolo[2,3-d]pyrimidin-5-yl)ethyl)benzamido)pentanedioate (I) prepared according to the present invention was observed to have potassium content ranging between 14.5% to 16.5% w/w on anhydrous basis. In a particular embodiment, it was observed as 15.7% potassium content on anhydrous basis. In one of our process using starting material as N-[4-[2-(2-amino-4,7-dihydro-4-oxo-1H-pyrrolo[2,3-d]pyrimidin-5-yl)ethyl]benzoyl]-L-glutamic dimethyl ester, no traces of sodium contents were located.

The compound of formula (I) according to the present invention is isolated as dipotassium salt of pemetrexed having purity greater than 99% w/w and total impurities not more than 0.7% w/w. Said compound of formula (I) and its hydrates are useful as active pharmaceutical ingredient in pharmaceutical compositions for the treatment of cancer.

Pemetrexed di potassium and its hydrates can be isolated by conventional processes, which are not limited to scrapping, breaking, triturating and if required conventional drying. In further aspect, the Pemetrexed di potassium and its hydrates obtained by the process of the present application may be formulated as solid compositions for oral administration in the form of capsules, tablets, pills, powders or granules useful in the treatment or prevention of cancer.

Pemetrexed di potassium and its hydrates of the present invention may have one or more advantageous and desirable properties compared to the known Pemetrexed di sodium, which are not limited to better stability, solubility and quality parameter leading to improved storage and distribution.

The Pemetrexed di potassium and its hydrates described herein were characterized by NMR (Jeol 400 MHz)/IR (Make—Perkin Elmer, model-spectrum 100) and HPLC (Waters Alliance using Empower software). While determining purity by HPLC, following analytical chromatographic conditions were used to determine the purity of the material along with impurity profile wherever required. Certain conditions may vary to some extent as per system suitability as well as person skilled in the art to perform the sample preparation and executing the analysis, however, following method of purity according to the one of the merits of invention provides robust, consistent and reliable information.

Column: 4.6×250 mm, 5p. packing L-1 or equivalent.
Flow rate: 1.0 mL/min.

### Detector: 226 nm

Injection volume: 10
Column Oven temperature: 25° C.±2° C.
Mobile phase-A: Buffer—0.05M KH₂PO₄ in 1000 mL water adjust pH to 6.0
Mobile phase-B: HPLC grade Acetonitrile

### Gradient Program

| Time in minutes | Mobile Phase-A | Mobile Phase-B |
|---|---|---|
| 0 | 90 | 10 |
| 5.0 | 90 | 10 |
| 17.0 | 70 | 30 |
| 40.0 | 70 | 30 |
| 45.0 | 90 | 10 |
| 50.0 | 90 | 10 |

Illustrative analytical data for the Pemetrexed di potassium and its hydrates obtained in the Examples are set forth in the example section.

In another embodiment, the Pemetrexed di potassium and its hydrates obtained by the process of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerine, propylene glycol or liquid paraffin.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising Pemetrexed di potassium and its hydrates of the present application include, but are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions derived from Pemetrexed di potassium and its hydrates of the present application may also comprise to include the pharmaceutically acceptable carrier used for the preparation of solid dispersion, wherever utilized in the desired dosage form preparation.

Certain specific aspects and embodiments of the present application will be explained in more detail with reference to the following examples, which are provided by way of illustration only and should not be construed as limiting the scope of the invention in any manner.

### Example-1

### Preparation of N-[4-[2-(2-amino-4,7-dihydro-4-oxo-1H-pyrrolo[2,3-d]pyrimidin-5-yl)ethyl]benzoyl]-L-glutamic diacid dipotassium salt from Pemetrexed disodium salt

To a mixture of N-[4-[2-(2-amino-4,7-dihydro-4-oxo-1H-pyrrolo[2,3-d]pyrimidin-5-yl)ethyl]benzoyl]-L-glutamic disodium salt (25.0 g, 0.05 mol) and DM water (375 ml) was added slowly aqueous solution −17% of hydrochloric acid at temperature 20-25° C. to adjust the pH 2.5-3.5 under stirring and the mixture was continued to stir at 20-25° C. for 3 hour. Reaction mass was filtered and washed with 50 ml of DM water. Suck dried for 1 hour. Suck dried solid (approx. 20.0 g) was suspended in DM water (40 ml) and cooled to 0-10° C. under stirring. Added slowly aqueous solution of 7.8 g KOH in 40 ml of DM water under stirring at 0-10° C. and reaction mass was stirred for 1 hour at 0-10° C. pH of reaction mass was adjusted to 7.5-8.5 by using −17% hydrochloric acid solution. Activated carbon (2.5 gm) added to reaction mass and stirred for 15 min. Reaction mass filtered and residue washed with 20 ml of DM water. Cool the filtrate to 0-10° C. under stirring for 1 hour. Add slowly the ethanol (320 ml) within 60-80 mins at 0-10° C. under stirring. Temperature is raised to 20-25° C. slowly under stirring and maintained for 3-4 hours at 20-25° C. Solid filtered, washed with ethyl alcohol (50 ml) and dried to give the title compound (22 g, 39.79%)

HPLC Purity: 99.39% w/w

Moisture content: 11.42% w/w

¹H NMR (D₂O, 400 MHz): δ ppm 2.03-2.10: m, 1H, —CH₂, 2.18-2.22: m, 1H, —CH₂, 2.32-2.36: t, 21-1, —CH₂, 2.87: s, 4H, 2×—CH₂, 4.34-4.37: m, 1H, —CH, 6.37: s, 1H, —CH, 7.20-7.23: d, 2H, 2×—CH (Ar), 7.67-7.69: d, 2H, 2×CH (Ar) ¹³C NMR (D₂O, 100 MHz): 8 ppm 182.988 (COOK), 179.785 (COOK), 170.507 (CO—NH), 161.583 (CO—NH, pyrimidine ring), 152.469 (C), 151.243 (C), 147.291 (C), 131.179 (C), 128.899 (C), 127.606 (C), 118.863 (CH), 115.669 (CH), 99.442 (C), 56.723 (CH), 35.845 (CH₂), 35.046 (CH₂), 29.160 (CH₂), 27.423 (CH₂)

### Example-2

### Preparation of N-[4-[2-(2-amino-4,7-dihydro-4-oxo-1H-pyrrolo[2,3-d]pyrimidin-5-yl)ethyl]benzoyl]-L-glutamic diacid dipotassium salt from Pemetrexed diacid

To a mixture of N-[4-[2-(2-amino-4,7-dihydro-4-oxo-1H-pyrrolo[2,3-d]pyrimidin-5-yl)ethyl]benzoyl]-L-glutamic di-acid (45.0 g, 0.10 mol) and DM water (180 ml) was added slowly aqueous solution of 17.72 g of potassium hydroxide in 45 ml of DM water at 0-5° C. under stirring and the mixture was continued to stir at 0-5° C. for 1 hour after complete addition. Now pH of aqueous layer was adjusted to 7.5-8.5 by using 17.5% hydrochloric acid solution. Activated carbon (4.5 gm) added to reaction mass and stirred for 15 min. Reaction mass filtered and residue washed with 20 ml of DM water. Cool the filtrate to 0-10° C. under stirring for 1 hour. Added slowly the filtered ethanol (1.1 L) within 60-80 mins at 0-10° C. under stirring. Temperature raised to 20-25° C. slowly under stirring and maintained for 3-4 hours at 20-25° C. Solid filtered, washed with ethyl alcohol (50 ml) and dried under vacuum to give the title compound (38 g, 71.67%) with purity of 99.60%. Moisture content: 10.95%.

### Example-3A

### Preparation of N-[4-[2-(2-amino-4,7-dihydro-4-oxo-1H-pyrrolo[2,3-d]pyrimidin-5-yl)ethyl]benzoyl]-L-glutamic diacid dipotassium salt from Pemetrexed dimethyl ester

### Process for Preparing Pemetrexed Dimethyl Ester:

To a mixture of 4-(2-(2-amino-4-oxo-4,7-dihydro-3H-pyrrolo[2,3-d]pyrimidin-5-yl)ethyl)benzoic acid (200 g, 0.67 mol) and 2-chloro-4,6-dimethoxy-1,3,5-triazine (129.4 g, 0.74 mol) in dimethylformamide was added N-methylmorpholine (203.46 g, 2.01 mol) at 0-5° C. under stirring and the mixture was stirred at 0-5° C. for 1.5 hour. Added slowly the solution of L-glutamic acid dimethylester hydrochloride (206.0 g, 0.97 mol) in dimethylformamide at 0-5° C. under stirring and the mixture maintained at 25-30° C. for 2-3 hours, when the reaction was found to be complete. The reaction mixture was poured into 4000 ml of DM water followed by addition of potassium bicarbonate and dichloromethane. The dichloromethane layer separated and washed successively two times with water (4 L each). The dichloromethane layer was evaporated to dryness under reduced pressure. The residue was dissolved in isopropyl alcohol (1 L) at 80-85° C. under stirring. Cooled the dissolved solution slowly to rt under stirring. The mass was stirred at rt for 3-4 hours and solid was filtered, washed with isopropyl alcohol (200 ml) and dried to give the title compound (200 g, 65.5%) with purity of 98.56%.

### Process for Preparing Pemetrexed Dipotassium

The process for preparing pemetrexed dipotassium salt comprise of following sequence of operations—

To a mixture of N-[4-[2-(2-amino-4,7-dihydro-4-oxo-1H-pyrrolo[2,3-d]pyrimidin-5-yl)ethyl]benzoyl]-L-glutamic dimethyl ester (50.0 g, 0.11 mol) and dichloromethane (500 ml) was added slowly aqueous solution of 18.47 g of potassium hydroxide in 200 ml of DM water at 0-5° C. under stirring and the mixture was continued to stir at 0-5° C. for 1 hour after complete addition. Dichloromethane layer was separated. Fresh dichloromethane (100 ml) was added to aqueous layer and separated after 15 min stirring and settling each. Dichloromethane layer separated. Now pH of aqueous layer was adjusted to 7.5-8.5 by using 17% hydrochloric acid solution. Activated carbon (5 gm) added to reaction mass and stirred for 15 min. Reaction mass filtered and residue washed with 50 ml of DM water. Cool the filtrate to 0-10° C. under stirring. Add into slowly the ethanol (1.2 L) within 60-80 minutes at 0-10° C. under stirring. Temperature was raised to 20-25° C. slowly under stirring and maintained for 3-4 hours at 20-25° C. Solid filtered, washed with ethyl alcohol (50 ml) and dried to give the title compound (22 g, 39.79%) with purity of 99.39%. Moisture content: 11.10%

### Example-3B

### Preparation of N-[4-[2-(2-amino-4,7-dihydro-4-oxo-1H-pyrrolo[2,3-d]pyrimidin-5-yl)ethyl]benzoyl]-L-glutamic diacid dipotassium salt from Pemetrexed dimethyl ester

500 ml of dimethylformamide and 100 g 4-(2-(2-amino-4-oxo-4,7-dihydro-3H-pyrrolo[2,3-d]pyrimidin-5-yl)ethyl)benzoic acid were charged into the reaction flask at 25-30° C. under nitrogen atmosphere. After stirring for 30 minutes, 64.7 g of 2-chloro-4,6-dimethoxy-1,3,5-triazine was added to the reaction mixture. Then the reaction mixture was cooled to 0-5° C. and slowly (within 45-60 min) 101.73 g of N-methylmorpholine was added. The reaction mixture was stirred for 90 mins and then 103.0 g solution of L-glutamic acid dimethylester hydrochloride in 200 ml of dimethylformamide was added drop-wise in 1.5-2 hrs under stirring. Further stirred the reaction mass for 1 hr. Cooling was removed and the reaction was allowed to come to 25-30° C. wherein stirring is performed for ˜3 hrs. After completion of the reaction as monitored by HPLC the reaction mixture was poured into 2000 ml of DM Water under stirring. pH of the reaction mixture was adjusted to 7.5 to 8.0 by adding 40.0 g solid potassium bicarbonate slowly. 2000 ml of dichloromethane was added and stirring was done for 15 minutes. Then the different solvent layers were allowed to settle and separate. DCM layer was washed with 2×2000 ml of DM Water and the DCM layer was taken in RB flask and cooled to 15-20° C. under stirring.

65.83 g of KOH solution in 400 ml of DM Water was added to the DCM layer slowly within 30 minutes under stirring. Stirring was performed for 60 minutes after complete addition of KOH solution. Solvent layers were separated and the product containing aqueous layer was washed with 500 ml of dichloromethane. The dichloromethane layers were separated and aqueous layer was charged into RB Flask under stirring at 15-20° C. The pH of reaction mixture was adjusted to 7.5-8.0 with 17% HCl solution and at room temperature 10 g activated Carbon was added to the reaction mixture. After stirring for 15 mins the reaction mixture was filtered through celite bed.

To the filtrate was added 350 g of potassium acetate solution in 550 ml DM water at 15-20° C. under stirring. Further stirring was carried out for 3-4 hours at 0-10° C. The solid obtained was filtered and suck dried for half an hour. The solid material was then washed with 200 mL of ethanol followed by suck drying for 30 minutes. The wet material was then dried under vacuum at 50-55° C. for 8-10 hrs.

820 ml of purified water was charged into a 3 ltr RB flask at room temperature under stirring. 82.0 g dried material obtained above, was added to the reaction mixture at room temperature under stirring. After stirring for 15 minutes, pH of reaction mass was adjusted between 7.5 to 8.5 by using KOH (1N)/HCl (17%) solution to get a clear solution. The obtained clear solution was stirred for 25 to 30 minutes and 287.0 g of potassium acetate was added under stirring which was continued for 3 hrs. The reaction mass was filtered and suck dried for 30 minutes, followed by washing with 164 ml of ethanol. The wet material obtained was suck dried for 30 minutes, followed by further drying in vacuum tray drier (VTD) at 40-45° C. for 8-10 hrs under vacuum of 650 mmHg, to obtain the title compound (47 g, 90.38%) with purity of 99.66%.

1. Di potassium (S)-2-(4-(2-(2-amino-4-oxo-4,7-dihydro-3H-pyrrolo[2,3-d]pyrimidin-5-yl)ethyl)benzamido)pentanedioate (I) 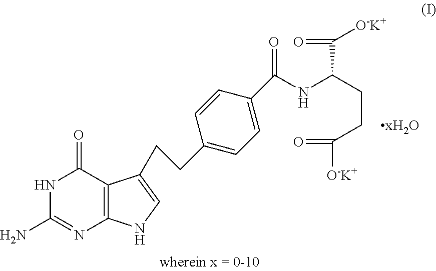
and its hydrates.

2. A process for preparing Di potassium (S)-2-(4-(2-(2-amino-4-oxo-4,7-dihydro-3H-pyrrolo[2,3-d]pyrimidin-5-yl)ethyl)benzamido)pentanedioate (I) comprising the steps of—
a) providing a solution of compound of Formula (A); 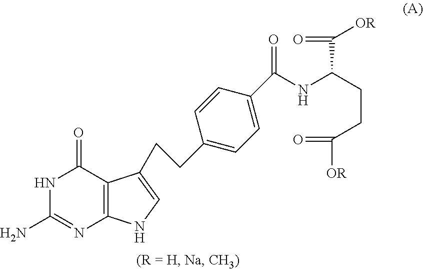
b) cooling the reaction mass between 0-15° C.;
c) adding aqueous solution of KOH under stirring at 0-15° C.;
d) stirring the reaction mass for 30-120 mins at 0-25° C.;
e) adjusting pH of aqueous reaction mass to 7.0-8.5 by using aqueous hydrochloric acid solution;
f) optionally performing the carbon treatment;
g) optionally adding a water soluble potassium salt to the reaction mixture;
h) cooling the reaction mass to 0-10° C. under stirring;
i) adding alcohol solvent at 0-10° C.;
j) optionally repeating the steps a) to g) at room temperature, by isolating the solid material from step i); and
k) isolating the solid product of formula (I).

3. Process for preparing Di potassium (S)-2-(4-(2-(2-amino-4-oxo-4,7-dihydro-3H-pyrrolo[2,3-d]pyrimidin-5-yl)ethyl)benzamido)pentanedioate (I), according to claim 2, wherein in step a) when R is Na or H, compound of Formula (A) is provided as solution in water solvent optionally containing hydrochloric acid; and when R is CH₃ compound of Formula (A) is provided as solution in a water immiscible organic solvent selected from dichloromethane, toluene, hexane and heptane.

4. A process for preparing compound of Formula (I) according to claim 2, wherein water soluble potassium salt used in step g) is potassium acetate.

5. A process for preparing compound of Formula (I) according to claim 2, alcohol used in step i) is selected from C1 to C5 alcohol.

6. A process for preparing compound of formula (I) according to claim 2, wherein step m) of isolating the solid product of formula (I) comprise of—
a) filtering the solid;
b) washing with alcohol (C1-05) solvent; and
c) drying the solid to get compound of formula:—(I) having purity greater than 99% w/w.

7. A process according to claim 2, wherein KOH is used up to more than 2.2 Moles but less than 4.0 moles with respect to starting material i.e. compound of Formula (A)

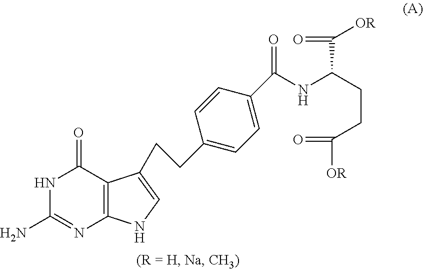

8. Di potassium (S)-2-(4-(2-(2-amino-4-oxo-4,7-dihydro-3H-pyrrolo[2,3-d]pyrimidin-5-yl)ethyl)benzamido)pentanedioate (I), wherein potassium content ranges between 14.5% to 16.5% w/w on anhydrous basis.

9. Di potassium (S)-2-(4-(2-(2-amino-4-oxo-4,7-dihydro-3H-pyrrolo[2,3-d]pyrimidin-5-yl)ethyl)benzamido)pentanedioate (I) according to claim 8, wherein total impurities are not more than 0.7% w/w.

10. A pharmaceutical composition comprising Di potassium (S)-2-(4-(2-(2-amino-4-oxo-4,7-dihydro-3H-pyrrolo[2,3-d]pyrimidin-5-yl)ethyl)benzamido)pentanedioate (I) according to claim 1 and a pharmaceutically acceptable amount of one or more excipients.
